(19) United States
(12) Patent Application Publication
(10) Pub. No.: US 2024/0052016 A1
(43) Pub. Date: Feb. 15, 2024

(54) HUMAN IL-10 RECEPTOR ALPHA FUSION PROTEINS

(71) Applicant: EpicentRX, Inc., La Jolla, CA (US)

(72) Inventors: Christopher LARSON, San Diego, CA (US); Tony R. Reid, San Diego, CA (US); Bryn T. Oronsky, Los Altos Hills, CA (US)

(21) Appl. No.: 18/494,508

(22) Filed: Oct. 25, 2023

Related U.S. Application Data
(62) Division of application No. 16/651,079, filed on Mar. 26, 2020, now Pat. No. 11,834,492.
(60) Provisional application No. 62/564,145, filed on Sep. 27, 2017.

(51) Int. Cl. C07K 14/715; A61P 35/00; C07K 14/54; C12N 15/62

(52) U.S. Cl. CPC C07K 14/7155; A61P 35/00; C07K 14/5428; C12N 15/62; A61K 38/00

(57) ABSTRACT

Provided is a fusion protein, e.g., a cytokine receptor fusion protein, e.g., an IL-10 trap, with a novel linker sequence to permit the fusion protein to functionally optimally, e.g., to permit a cytokine receptor portion of a cytokine receptor fusion protein to bind optimally to its target cytokine. The fusion protein, or an expression vector encoding for the fusion proteins, can be used to treat cell proliferative diseases and disorders, including certain forms of cancer and inflammatory disorders.

### CROSS REFERENCE TO RELATED APPLICATIONS

This application is a Divisional of U.S. patent application Ser. No. 16/651,079, filed Mar. 26, 2020, which is the National Stage of International (PCT) Application No. PCT/US2018/053197, filed Sep. 27, 2018, which claims priority to U.S. Provisional Patent Application No. 62/564,145, filed Sep. 27, 2017, all of which are hereby incorporated by reference in the present disclosure in their entirety.

### STATEMENT REGARDING SEQUENCE LISTING

The contents of the electronic sequence listing (203592001610seqlistxml; Size: 129,634 bytes; and Date of Creation: Oct. 25, 2023) is herein incorporated by reference in its entirety.

### FIELD OF THE INVENTION

The field of the invention is molecular biology, specifically immunology and fusion proteins, e.g., cytokine receptor fusion proteins.

### BACKGROUND

Cytokines are small, secreted cell signaling proteins that have a wide range of activities including regulation of cell growth and differentiation and modulation of immune function. Cytokines, cytokine receptors, and certain other immunomodulatory proteins have been used as therapeutics to treat a variety of medical conditions. However, the administration of such proteins, for example, by subcutaneous or vascular routes, can result in inappropriate cellular and extracellular localization thereby limiting therapeutic activity and/or increasing the risk of toxicity.

IL-10 is a homodimeric cytokine with immunoregulatory properties produced by cells including activated Th2 cells, B cells, keratinocytes, monocytes and macrophages (Moore et al. (1993) ANNU. REV. IMMUNOL. 11:165). IL-10 inhibits activation and effector functions of a variety of cells including T cells, monocytes and macrophages. In particular, IL-10 inhibits cytokine synthesis, including that of IL-1, IFN-γ, and TNF, by cells such as Th1 cells, natural killer cells, monocytes, and macrophages (Fiorentino et al. (1989) J. EXP. MED. 170:2081-2095; Fiorentino et al. (1991) J. IMMUNOL. 146:3444; Hsu et al. (1992) INT. IMMUNOL. 4:563; Hsu et al. (1992) INT. IMMUNOL. 4:563; D′Andrea et al. (1993) J. EXP. MED. 178:1041; de Waal Malefyt et al. (1991) J. EXP. MED. 174:915; Fiorentino et al. (1991) J. IMMUNOL. 147:3815). Multiple pathogens, including intracellular pathogens, elicit IL-10 production to slow or stall the effective removal of the pathogen by the immune system (Moore et al. (1993) supra).

Despite the advances that have been made to date in treating IL-10 mediated disorders, there is a need for improved therapies for treating such disorders.

### SUMMARY OF THE INVENTION

The invention is based, in part, upon the discovery of linker sequences that improve the function of fusion proteins, e.g., cytokine receptor fusion proteins, e.g., IL-10 receptor (IL-10R) fusion proteins, e.g., IL-10 receptor subunit alpha (IL-10RA) fusion proteins, e.g., IL-10 traps. The linker sequences may permit a ligand binding portion of a fusion protein (e.g., a cytokine receptor) to bind optimally to a ligand (e.g., a cytokine), provide temporal and spatial colocalization of two or more components of a fusion protein (e.g., two subunits of a dimeric cytokine), optimize expression from an expression vector (e.g., a viral vector), reduce immunogenicity, or provide a cleavage site to allow for release of a component of the fusion protein. For example, the linker sequences may provide sufficient flexibility to allow a ligand binding domain of a cytokine receptor to adopt a native conformation in the context of a fusion protein, and minimize the potential immunogenicity of the fusion protein for use as a therapeutic agent.

In one aspect, the invention provides an isolated fusion protein that comprises, for example, in an N- to C-terminal orientation: a first portion of an extracellular domain, transmembrane domain, or intracellular domain of a cytokine, cytokine receptor, or immunomodulatory protein; an amino acid linker; and at least one of, a second portion of an extracellular domain, transmembrane domain, or intracellular domain of a cytokine, cytokine receptor, or immunomodulatory protein; an immunoglobulin (Ig) hinge region; and an immunoglobulin (Ig) Fc domain. In certain embodiments, the linker comprises from about 5 to about 40 amino acid residues. In certain embodiments, the fusion protein comprises a portion of an IL-10 receptor, e.g., a human IL-10 receptor, e.g., IL-10RA.

In another aspect, the invention provides an isolated fusion protein that comprises, in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues. In certain embodiments, the cytokine receptor is an IL-10 receptor, e.g., a human IL-10 receptor, e.g., IL-10RA.

In certain embodiments of any of the foregoing fusion proteins, the amino acid linker may comprise, e.g., from about 5 to about 15, from about 5 to about 20, from about 5 to about 30, from about 10 to about 15, from about 10 to about 20, from about 10 to about 30, from about 10 to about 40, from about 15 to about 20, from about 15 to about 30, or from about 15 to about 40 amino acid residues.

In certain embodiments of any of the foregoing fusion proteins, the amino acid linker sequence is derived from an endogenous human protein, e.g., IgG1, IgG2, IgG3, IgG4, IgA1, IgA2, IgD, IgE, IgM, albumin, or casein. In certain embodiments, the amino acid linker comprises a C-terminal portion of an immunoglobulin (Ig) CH1 domain, e.g., an IgG1, IgG2, IgG3, IgG4, IgA1, IgA2, IgD, IgE, or IgM CH1 domain. In certain embodiments, the amino acid linker comprises an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 53, SEQ ID NO: 54, and SEQ ID NO: 57. In certain embodiments, the amino acid linker comprises a C-terminal portion of an IgG1 CH1 domain, e.g., the amino acid linker comprises an amino acid sequence selected from SEQ ID NO: 1, SEQ ID NO: 53, SEQ ID NO: 54, and SEQ ID NO: 57, e.g., the amino acid sequence of SEQ ID NO: 57.

In certain embodiments of any of the foregoing fusion proteins, the amino acid linker comprises a sequence derived from a cytokine, signaling molecule, immunomodulatory protein or peptide, or a biologically active peptide.

In certain embodiments of any of the foregoing fusion proteins, the amino acid linker comprises a cleavage site, e.g., a proteolytic cleavage site, e.g., a proteolytic cleavage site that is cleaved by a protease present in the endoplasmic reticulum or golgi of a eukaryotic cell. In certain embodiments, the proteolytic cleavage site is a furin cleavage site, e.g., a furin cleavage site comprising the sequence RX₁X₂R (SEQ ID NO: 50), wherein X₁ is any amino acid, and X₂ is Lys or Arg, e.g., a furin cleavage site comprising the sequence RAKR (SEQ ID NO: 51). In certain embodiments of any of the foregoing fusion proteins, the amino acid linker is proteolytically stable in a mammal or plant.

In certain embodiments of any of the foregoing fusion proteins, the soluble portion of an extracellular domain of a cytokine receptor is a soluble portion of an extracellular domain of the human IL-10R, e.g., IL-10RA. For example, in certain embodiments, the soluble portion of an extracellular domain of a cytokine receptor comprises the amino acid sequence of SEQ ID NO: 12 or amino acid residues 22-229 of SEQ ID NO: 12.

In certain embodiments of any of the foregoing fusion proteins, the fusion protein comprises one or more of IL-10, TGF-β, a TGFβ receptor, e.g., the TGFβ type II receptor (TβRII), CD80, CD19, CD20, IL-1, IL-2, IL-3, IL-4, IL-5, IL-6, IL-7, IL-8, IL-9, IL-12B/p40, IL-23A/p19, IL27A/p28, IL-27B/EBI3, IL-15, CD154, CD70, TNF-alpha, CD86, CD137, CD137L, BORIS/CTCFL, FGF, ICAM, IL-24, GM-CSF, MAGE, NY-ESO-1, angiostatin, endostatin, acetylcholine, interferon-gamma, DKK1/Wnt, p53, Ox40L, GM-CSF, an IL-15 receptor fusion protein, GITRL, CD40L, CD70, secreted flagellin, IL-12, thymidine kinase, an anti-PD-1 antibody heavy chain or light chain, an anti-PD-L1 antibody heavy chain or light chain, and an anti-CTLA-4 antibody heavy chain or light chain, or a functional fragment thereof.

In certain embodiments of any of the foregoing fusion proteins, the Ig hinge region is selected from an IgG1, IgG2, IgG3, IgG4, IgA1, IgA2, IgD, IgE, and IgM hinge region, and the Ig Fc domain, is selected from IgG1, IgG2, IgG3, IgG4, IgA1, IgA2, IgD, IgE, and IgM Fc domain In certain embodiments, the Ig hinge region and Fc domain together comprise an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21. In certain embodiments, the Ig Fc, Ig hinge region, and Ig CH1 domain are derived from a single immunoglobulin.

In certain embodiments of any of the foregoing fusion proteins, the fusion protein comprises an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 55, SEQ ID NO: 56, and SEQ ID NO: 58. In certain embodiments, the fusion protein comprises an amino acid sequence selected from SEQ ID NO: 22, SEQ ID NO: 55, SEQ ID NO: 56, and SEQ ID NO: 58. In certain embodiments, the fusion protein comprises the amino acid sequence of SEQ ID NO: 58.

In certain embodiments of any of the foregoing fusion proteins, the fusion protein comprises an amino acid sequence having greater than 80%, 85%, 90%, 95%, 96%, 97%, 98% or 99% sequence identity to a sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 55, SEQ ID NO: 56, and SEQ ID NO: 58.

In another aspect, the invention provides a dimeric cytokine binding protein comprising two of any of the foregoing fusion proteins covalently linked together, wherein each fusion protein comprises an extracellular domain of a cytokine receptor, and wherein the two extracellular domains together define a binding site for a cytokine.

In another aspect, the invention provides a nucleic acid comprising a nucleotide sequence that encodes for any of the foregoing fusion proteins.

In another aspect, the invention provides an expression vector comprising any of the foregoing nucleic acids.

In another aspect, the invention provides a host cell comprising any of the foregoing the expression vectors. In another aspect, the invention provides a method of producing a fusion protein comprising growing a host cell under conditions to express the fusion protein and purifying the fusion protein. In another aspect, the invention provides a method of expressing a fusion protein in a target cell comprising exposing the cell to an effective amount of any of the foregoing expression vectors. In certain embodiments, the fusion protein is cleaved posttranslationally into two polypeptide chains.

In another aspect, any of foregoing fusion proteins or expression vectors can be used, e.g., to reduce cytokine activity in a subject, thereby treating various medical indications that are mediated by a cytokine, for example, IL-10. In another aspect, any of the foregoing fusion proteins or expression vectors can be used to inhibit proliferation of tumor cells in vitro and/or in vivo, inhibit tumor growth in a subject in need thereof, or treat cancer in a subject in need thereof. The subject may be, e.g., an animal, e.g., a mammal, e.g., a human, e.g., a pediatric human For example, when administered to a human subject with cancer, the fusion proteins or expression vectors inhibit or reduce tumor growth, or, reduce the tumor load, in the subject.

In certain embodiments, the cancer may be selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, parathyroid cancer (e.g., parathyroid carcinoma), endometrial cancer, neuroendocrine cancer, lymphoma (e.g., Hodgkin and non-Hodgkin), leukemia, merkel cell carcinoma, gastrointestinal stromal tumors, multiple myeloma, uterine cancer, a sarcoma, kidney cancer, ocular cancer, pancreatic cancer, and a germ cell cancer (e.g., ovarian germ cell cancer). In certain embodiments, the cancer may be selected from leukemia, breast cancer, lung cancer, pancreatic cancer, endometrial cancer, ovarian cancer, prostate cancer, cervical cancer, brain cancer, skin cancer, colorectal cancer, gastric cancer, head and neck cancer, and leukemia.

In certain embodiments, the fusion protein or expression vector is administered in combination with one or more therapies selected from surgery, radiation, chemotherapy, immunotherapy, hormone therapy, and virotherapy. In certain embodiments, the fusion protein or expression vector is administered in combination with a lymphocyte, e.g., a T-cell, e.g., a CAR T-cell.

Any of the foregoing fusion proteins or expression vectors can also be used to treat an inflammatory condition or infection in a subject in need thereof.

These and other aspects and advantages of the invention are illustrated by the following figures, detailed description and claims.

### DESCRIPTION OF THE DRAWINGS

The invention can be more completely understood with reference to the following drawings.

FIG. 1A depicts a schematic of a dimeric cytokine receptor on the cell surface (left), an antibody (middle), and a receptor-Fc fusion that optimally binds a target cytokine (right).

FIG. 1B depicts a receptor-Fc fusion, e.g., a cytokine trap, that is sterically constrained from optimal binding to a target cytokine (left), or that adopts an optimal binding configuration (right).

FIG. 2 depicts a sequence alignment of the amino acid sequences of the human IgG1, IgG2, IgG3, IgG4, IgA1, IgA2, IgD, IgE, and IgM CH1 domains (top) and CH2 domains (bottom).

FIG. 3 depicts a Western blot for phosphorylated Stat3 following treatment of reporter cells with IL-10 and/or the IL-10RA fusion proteins IL-10R-IgG and IL-10R-Fc as indicated. Total Stat3 was used as a loading control. IL-10 activity was markedly reduced by IL-10R-IgG compared to IL-10R-Fc.

### DETAILED DESCRIPTION

The invention provides a recombinant fusion protein for use in the treatment of a variety of medical conditions, for example, in inhibiting proliferation of a tumor cell, inhibiting tumor growth, treating cancer, treating an inflammatory condition, or treating an infection, in a subject. Exemplary fusion proteins comprise: a first portion of an extracellular domain, transmembrane domain, or intracellular domain of a cytokine, cytokine receptor, or immunomodulatory protein; an amino acid linker; and at least one of, a second portion of an extracellular domain, transmembrane domain, or intracellular domain of a cytokine, cytokine receptor, or immunomodulatory protein; an immunoglobulin (Ig) hinge region; or an immunoglobulin (Ig) Fc domain It is contemplated that the first and second portions can be portions of the same protein or portions of different proteins, and, even if the same protein, the first and second portions can be different portions of the same protein. In certain embodiments, the linker comprises from about 5 to about 40 amino acid residues. Exemplary fusion proteins of the invention include cytokine traps.

A cytokine trap, e.g. an IL-10 trap, is a molecule containing a soluble portion of the extracellular domain of a cytokine receptor, e.g., an IL-10 receptor (IL-10R), e.g., an IL-10 receptor alpha subunit (IL-10RA) designed to bind or otherwise sequester a target cytokine. In a cytokine trap, the extracellular domain of a cytokine receptor may be fused to an immunoglobulin (Ig) hinge region and immunoglobulin (Ig) Fc domain which can allow, e.g., for increased stability, Fc effector functions and/or multimerization, e.g., dimerization. Dimerization afforded by fusion to an Ig hinge region and Ig Fc domain is particularly advantageous for cytokine receptors that exist as dimeric receptor complexes on the cellular surface, such as, e.g., TβRII.

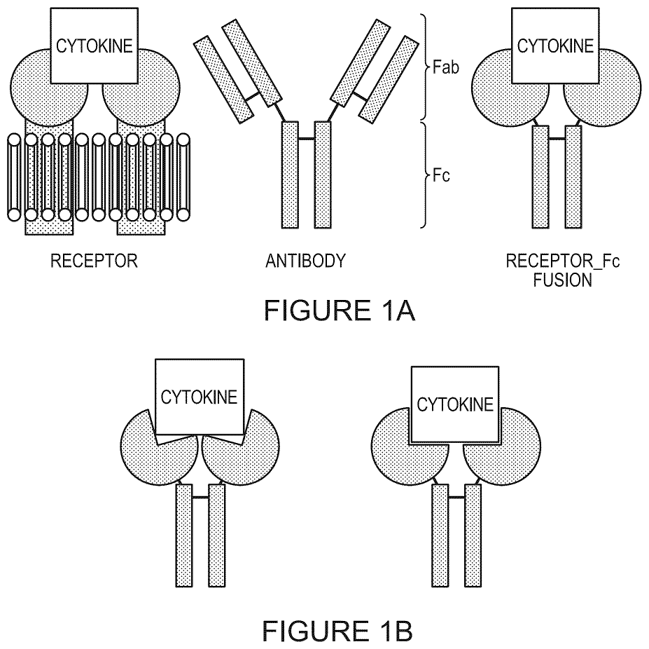

Conventional cytokine traps, e.g., IL-10 traps, comprise two polypeptide chains, each polypeptide chain comprising a soluble portion of an extracellular domain of a cytokine receptor fused to an Ig hinge region and an Ig Fc domain. The soluble portion of the extracellular domain of the cytokine receptor typically is fused directly to the Ig hinge region, without any intervening sequence. The two polypeptide chains are covalently linked by disulfide bonds between cysteine residues in each of the Ig hinge regions. Each polypeptide chain provides a soluble portion of an extracellular domain of a cytokine receptor, e.g., IL-10R, e.g., IL-10RA, and the two soluble portions of an extracellular domain of a cytokine receptor together define a binding site for a cytokine. A schematic representation of a dimeric cytokine receptor, an immunoglobulin (antibody) molecule, and a dimeric protein comprising two covalently linked fusion proteins each comprising a soluble portion of an extracellular domain of a cytokine receptor fused to an Ig hinge region and an Ig Fc domain is depicted in FIG. 1A.

The invention is based, in part, upon the discovery that conventional cytokine traps comprising a fusion protein of a soluble portion of an extracellular domain of a cytokine receptor to an Ig hinge region and Ig Fc domain, e.g., IL-10 traps, do not optimally bind their target cytokine. For example, a conventional IL-10 trap does not provide sufficient flexibility between the two IL-10 ligand binding domains to allow the two IL-10 ligand binding domains to come together in an optimal configuration to define an IL-10 binding site.

Thus, in one aspect, the invention provides an isolated fusion protein that comprises, in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues. The linker sequence allows, e.g., the binding domain in the extracellular domain of the cytokine receptor to bind optimally to its target cytokine. This is especially important when the cytokine binding protein is a dimer that comprises two of the foregoing fusion proteins that together define a binding site to bind the target cytokine. Without the linker, the two binding domains may be sterically constrained from forming the optimal binding site (FIG. 1B). Various features and aspects of the invention are discussed in more detail below.

### I. Fusion Proteins

Exemplary fusion proteins may comprise: a first portion of an extracellular domain, transmembrane domain, or intracellular domain of a cytokine, cytokine receptor, or immunomodulatory protein; an amino acid linker; and at least one of, a second portion of an extracellular domain, transmembrane domain, or intracellular domain of a cytokine, cytokine receptor, or immunomodulatory protein; an immunoglobulin (Ig) hinge region; and an immunoglobulin (Ig) Fc domain. For example, a disclosed fusion protein may comprise, in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues.

Exemplary cytokines include IL-1α, IL-1β, IL-18. IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF, and MSP.

As used herein, an “immunomodulatory” protein refers to a protein that modulates the function of the immune system of a subject Immunomodulatory proteins may, for example, modulate the function of, e.g., B-cells, T cells and/or the production of antibodies. Exemplary immunomodulatory proteins include checkpoint inhibitors. Exemplary immunomodulatory proteins may include, e.g., CTLA-4, CD70, IL-2, CD40L, OX40L, IL-12, IL-7, PD-1, or PD-L1, or any protein that modulates the activity thereof. Further exemplary immunomodulatory proteins may include an anti PD-1 antibody, or anti-PD-L1 antibody.

As used herein, a “soluble portion of an extracellular domain of a cytokine receptor” refers to any extracellular domain of a cytokine receptor or fragment of an extracellular domain of a cytokine receptor that is capable of binding to a target cytokine. It is understood that the soluble portion of an extracellular domain of a cytokine receptor also contemplates portions of the extracellular domain that comprise a binding domain that, either alone or in combination with a second binding domain (e.g., in the case of dimeric fusion proteins) is capable of binding to a target cytokine.

Exemplary cytokine receptors include type I cytokine receptors (e.g., GM-CSF receptors, G-CSF receptors, type I IL receptors, Epo receptors, LIF receptors, CNTF receptors, or TPO receptors), type II cytokine receptors (e.g., IL-10 receptors, IFN-alpha receptors (e.g., IFNAR1 or IFNAR2), IFN-beta receptors, IFN-gamma receptors (e.g., IFNGR1 or IFNGR2), chemokine receptors (e.g., CC chemokine receptors, CXC chemokine receptors, CX3C chemokine receptors, or XC chemokine receptors), tumor necrosis factor superfamily receptors (TNFRs; e.g., TNFRSF5/CD40, TNPRSF8/CD30, TNFRSF7/CD27, TNFRSF1A/TNFR1/CD120a, or TNPRSF1B/TNI-R2/CD120b), TGFβ superfamily receptors (e.g., TGFβ type I receptor or TGFβ type II receptor), or immunoglobulin (Ig) superfamily receptors (e.g., interleukin-1 receptors, CSF-1R, PDGFR (e.g., PDGFRA or PDGFRB), or SCFR). Preferred cytokine receptors include dimeric cytokine receptors, e.g., TGFβ superfamily receptors, e.g., the human TGFβ type II receptor (TβRII). In certain embodiments, the soluble portion of an extracellular domain of a cytokine receptor is a soluble portion of an extracellular domain of the human IL-10R, e.g., human IL-10RA, e.g., comprising the amino acid sequence of SEQ ID NO: 12, or an amino acid sequence having greater than 85%, 90%, 95%, 96%, 97%, 98% or 99% sequence identity to SEQ ID NO: 12, and/or a fragment thereof that comprises a binding domain that binds to IL-10.

The soluble portion of the extracellular domain of a cytokine receptor retains its ability to bind its native ligand. In certain embodiments, the soluble portion of the extracellular domain retains at least 50%, 60%, 70%, 80%, 90%, or 95% of the binding activity to its native ligand when compared to the full length cytokine receptor.

In certain embodiments, the fusion protein can comprise, e.g., one or more of TβRII, TGF-β, CD80, CD19, CD20, IL-1, IL-2, IL-3, IL-4, IL-5, IL-6, IL-7, IL-8, IL-9, IL-12B/p40, IL-23A/p19, IL-27A/p28, IL-27B/EB13, IL-15, CD154, CD70, TNF-alpha, CD86, CD137, CD137L, BORIS/CTCFL, FGF, ICAM, IL-24, GM-CSF, MAGE, NY-ESO-1, angiostatin, endostatin, acetylcholine, interferon-gamma, DKK1/Wnt, p53, Ox40L, GM-CSF, an IL-15 receptor fusion protein, GITRL, CD40L, CD70, secreted flagellin, IL-12, thymidine kinase, an anti-PD-1 antibody heavy chain or light chain, an anti-PD-L1 antibody heavy chain or light chain, and an anti-CTLA-4 antibody heavy chain or light chain, or a functional fragment thereof.

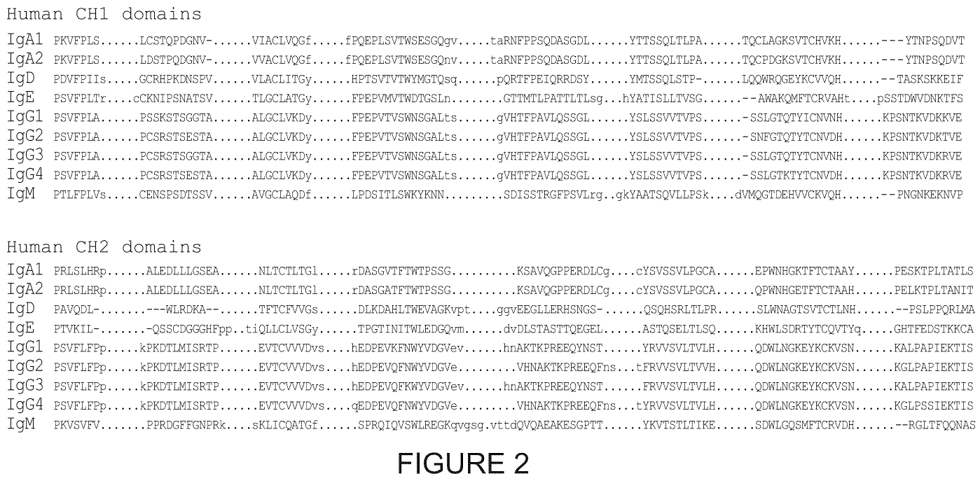

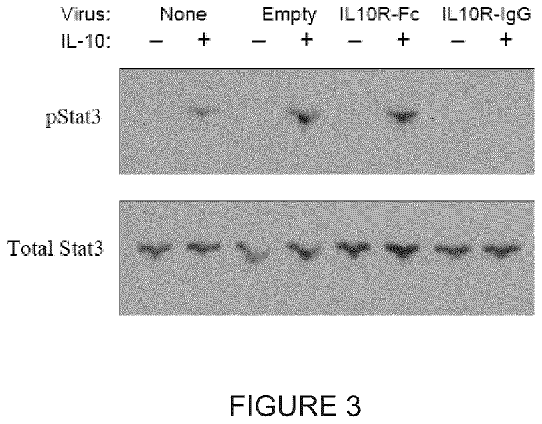

As used herein, the term “immunoglobulin (Ig) hinge region” refers to the amino acid sequence that typically connects CH1 and CH2 domains of an immunoglobulin heavy chain constant region. An Ig hinge region may include, e.g., one or more cysteine residues capable of forming disulfide bonds with cysteine residues in another protein chain. As used herein, the term “immunoglobulin (Ig) Fc domain” refers to a fragment of an immunoglobulin heavy chain constant region that is capable of binding to an Fc receptor. An Ig Fc domain may include, e.g., an immunoglobulin (Ig) CH2 and CH3 domain Boundaries between Ig CH1, CH2, and CH3 domains are well known in the art, and can be found, e.g., in the PROSITE database (available on the world wide web at prosite.expasy.org). For clarity, alignments of the amino acid sequences of the human IgG1, IgG2, IgG3, IgG4, IgA1, IgA2, IgD, IgE, and IgM CH1 and CH2 domains are provided in FIG. 2.

In certain embodiments, the Ig hinge region is selected from an IgG1, IgG2, IgG3, IgG4, IgA1, IgA2, IgD, IgE, and IgM hinge region, and the Ig Fc domain, is selected from an IgG1, IgG2, IgG3, IgG4, IgA1, IgA2, IgD, IgE, and IgM Fc domain In certain embodiments, the Ig hinge region and Fc domain together comprise an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21. In certain embodiments, the Ig hinge region and Fc domain together comprise an amino acid sequence having greater than 85%, 90%, 95%, 96%, 97%, 98% or 99% identity with a sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

The amino acid linker may permit a ligand binding portion of a fusion protein (e.g., a cytokine receptor) to bind optimally to a ligand (e.g., a cytokine), provide temporal and spatial co-localization of two or more components of a fusion protein (e.g., two subunits of a dimeric cytokine), optimize expression from an expression vector (e.g., a viral vector), reduce immunogenicity, or provide a cleavage site to allow for release of a component of the fusion protein.

The amino acid linker may comprise, e.g., from about 5 to about 15, from about 5 to about 20, from about 5 to about 25, from about 5 to about 30, from about 5 to about 35, from about 5 to about 40, from about 10 to about 15, from about 10 to about 20, from about 10 to about 25, from about 10 to about 30, from about 10 to about 35, from about 10 to about 40, from about 15 to about 20, from about 15 to about 25, from about 15 to about 30, from about 15 to about 35, or from about 15 to about 40 amino acid residues. The amino acids in the linker can be naturally occurring amino acids or modified amino acids.

In certain embodiments, the amino acid linker sequence is derived from an endogenous human protein, e.g., IgG1, IgG2, IgG3, IgG4, IgA1, IgA2, IgD, IgE, IgM, albumin, or casein. In certain embodiments, the amino acid linker comprises a C-terminal portion, for example, from about 5 to about 40 amino acids, of an immunoglobulin (Ig) CH1 domain, e.g., an IgG1, IgG2, IgG3, IgG4, IgA1, IgA2, IgD, IgE, or IgM CH1 domain. In certain embodiments, the amino acid linker comprises an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9. SEQ ID NO: 10, SEQ ID NO: 11, SEQ ID NO: 53, SEQ ID NO: 54, SEQ ID NO: 57, SEQ ID NO: 59, SEQ ID NO: 60, SEQ ID NO: 61, SEQ ID NO: 62, SEQ ID NO: 63, and SEQ ID NO: 64. In certain embodiments, the amino acid linker comprises a sequence having greater than 85%, 90%, 95%, 96%, 97%, 98% or 99% sequence identity to an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9. SEQ ID NO: 10, SEQ ID NO: 11, SEQ ID NO: 53, SEQ ID NO: 54, SEQ ID NO: 57, SEQ ID NO: 59, SEQ ID NO: 60, SEQ ID NO: 61, SEQ ID NO: 62, SEQ ID NO: 63, and SEQ ID NO: 64.

A protein or polypeptide is “derived from” a reference protein or polypeptide if it comprises an amino acid sequence that is substantially similar to all or a corresponding portion of the wild-type amino acid sequence of the reference protein or polypeptide. In certain embodiments, a protein or polypeptide that is derived from a wild-type protein or polypeptide may have one or more amino acid substitutions relative to the wild-type protein or polypeptide. For example, it is contemplated that a protein or polypeptide that is derived from a wild-type protein or polypeptide may have greater than 70%, 75%, 80%, 85%, 90%, 95%, 96%, 97%, 98% or 99% sequence identity to the wild-type protein or polypeptide. Further, it is contemplated that a protein or polypeptide that is derived from a wild-type protein or polypeptide may contain on more conservative substitutions relative to the wild-type protein or polypeptide. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

In certain embodiments, the amino acid linker sequence is derived from a cytokine, signaling molecule, immunomodulatory protein or peptide, or a biologically active peptide.

Further contemplated linker sequences include glycine- and serine-rich linkers, e.g., (G₄S)₃ (SEQ ID NO: 49). Additional exemplary linker sequences are disclosed, e.g., in George et al. (2003) PROTEIN ENGINEERING 15:871-879 and U.S. Pat. Nos. 5,482,858 and 5,525,491.

In certain embodiments, the amino acid linker may comprise a cleavage site, e.g., a proteolytic or a non-proteolytic cleavage site. In certain embodiments, the proteolytic cleavage site is cleaved by a protease present in a specific tissue, organelle or intracellular compartment. In certain embodiments, the linker comprises a proteolytic cleavage site and two cysteine residues that result in a disulfide linkage following proteolytic cleavage. In certain embodiments, the proteolytic cleavage site is cleaved by a protease selected from a matrix metalloproteinase (MMP), furin, PC1, PC2, PC3, cathepsin B, proteinase 3, and caspase 3. In certain embodiments, the cleavage site is a proteolytic cleavage site that is cleaved by a protease that is present in the endoplasmic reticulum or golgi of a eukaryotic cell. In certain embodiments, the proteolytic cleavage site is a furin cleavage site. Furin is a protease that is ubiquitously expressed and is localized to the golgi, where it recognizes the consensus sequence RX₁X₂R (SEQ ID NO: 50), wherein X₁ is any amino acid, and X₂ is Lys or Arg, and cleaves after the final Arg. Furin plays a biological role in cleaving propeptides of proteins that are trafficked through the golgi. Accordingly, in certain embodiments the proteolytic cleavage site is a furin cleavage site comprising the sequence RX₁X₂R (SEQ ID NO: 50), wherein X₁ is any amino acid, and X₂ is Lys or Arg, e.g., a furin cleavage site comprising the sequence RAKR (SEQ ID NO: 51).

In certain embodiments, the Ig Fc, Ig hinge region, and Ig CH1 domain are derived from a single immunoglobulin.

In certain embodiments, the fusion protein comprises an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 55, SEQ ID NO: 56, and SEQ ID NO: 58. In certain embodiments, a disclosed fusion protein comprises an amino acid sequence having greater than 85%, 90%, 95%, 96%, 97%, 98% or 99% sequence identity to a sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 55, SEQ ID NO: 56, and SEQ ID NO: 58.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. Sci. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In one aspect the invention provides a cytokine binding protein comprising two fusion proteins, wherein each fusion protein comprises in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues, wherein the two fusion proteins are covalently linked together, and wherein the two extracellular domains together define a binding site for a cytokine.

The cytokine binding protein may comprise two of the foregoing fusion proteins covalently linked together, wherein each fusion protein comprises an extracellular domain of a cytokine receptor, and wherein the two extracellular domains together define a binding site for a cytokine. The fusion proteins may be covalently linked, e.g., by disulfide bonds between cysteine residues in the Ig hinge region of each fusion protein. In certain embodiments, the fusion proteins, either monomeric or multimeric (e.g., dimeric) retain at least 50%, 60%, 70%, 80%, 90%, or 95% of the binding activity of the target ligand when compared to the native, full length cytokine receptor.

In certain embodiments, a cytokine binding protein of the invention binds a cytokine with a K_{D} of 200 nM, 100 nM, 20 nM, 15 nM, 10 nM, 9 nM, 8 nM, 7 nM, 6 nM, 5 nM, 4 nM, 3 nM, 2 nM, 1 nM, 50 pM, 25 pM or lower. In certain embodiments, a cytokine binding protein of the invention binds a cytokine with a K_{D} of from 200 nM to 100 nM, from 200 nM to 20 nM, from 200 nM to 10 nM, from 200 nM to 5 nM, from 200 nM to 1 nM, from 200 nM to 50 pM, from 200 nM to 25 pM, from 100 nM to 20 nM, from 100 nM to 10 nM, from 100 nM to 5 nM, from 100 nM to 1 nM, from 100 nM to 50 pM, from 100 nM to 25 pM, from 20 nM to 10 nM, from 20 nM to 5 nM, from 20 nM to 1 nM, from 20 nM to 50 pM, from 20 nM to 25 pM, from 10 nM to 5 nM, from 10 nM to 1 nM, from 10 nM to 50 pM, from 10 nM to 25 pM, from 5 nM to 1 nM, from 5 nM to 50 pM, from 5 nM to 25 pM, from 1 nM to 50 pM, from 1 nM to 25 pM, or from 50 pM to 25 pM. In certain embodiments, a cytokine binding protein of the invention binds IL-10 with a K_{D} of 200 nM, 100 nM, 20 nM, 15 nM, 10 nM, 9 nM, 8 nM, 7 nM, 6 nM, 5 nM, 4 nM, 3 nM, 2 nM, 1 nM, 50 pM, 25 pM or lower. In certain embodiments, a cytokine binding protein of the invention binds IL-10 with a K_{D} of from 200 nM to 100 nM, from 200 nM to 20 nM, from 200 nM to 10 nM, from 200 nM to 5 nM, from 200 nM to 1 nM, from 200 nM to 50 pM, from 200 nM to 25 pM, from 100 nM to 20 nM, from 100 nM to 10 nM, from 100 nM to 5 nM, from 100 nM to 1 nM, from 100 nM to 50 pM, from 100 nM to 25 pM, 20 nM to 10 nM, from 20 nM to 5 nM, from 20 nM to 1 nM, from 20 nM to 50 pM, from 20 nM to 25 pM, from 10 nM to 5 nM, from 10 nM to 1 nM, from 10 nM to 50 pM, from 10 nM to 25 pM, from 5 nM to 1 nM, from 5 nM to 50 pM, from 5 nM to 25 pM, from 1 nM to 50 pM, from 1 nM to 25 pM, or from 50 pM to 25 pM. K_{D} values may be determined by methods well known in the art, including surface plasmon resonance or bio-layer interferometry methods.

Exemplary fusion proteins of the invention include proteins comprising an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 55, SEQ ID NO: 56, and SEQ ID NO: 58. For clarity, the sequences of the individual elements of these proteins, and the proteins from which the sequences of the individual elements were derived, including the soluble portion of an extracellular domain of a cytokine receptor, the amino acid linker, the Ig hinge region, and the Ig Fc domain, are set forth in TABLE 1.

TABLE 1

| | | | Ig Hinge/ |
|---|---|---|---|
| | | | Ig Fc Source |
| | Receptor Source | Linker Source | Ig Hinge/ |
| Protein | Receptor SEQ ID | Linker SEQ ID | Ig Fc SEQ ID |
| SEQ ID | IL-10RA | IgG1 CH1 domain | IgG1 |
| NO: 22 | SEQ ID NO: 12 | SEQ ID NO: 1 | SEQ ID NO: 13 |
| SEQ ID | IL-10RA | IgG1 CH1 domain | IgG1 |
| NO: 55 | SEQ ID NO: 12 | SEQ ID NO: 53 | SEQ ID NO: 13 |
| SEQ ID | IL-10RA | IgG1 CH1 domain | IgG1 |
| NO: 56 | SEQ ID NO: 12 | SEQ ID NO: 54 | SEQ ID NO: 13 |
| SEQ ID | IL-10RA | IgG1 CH1 domain | IgG1 |
| NO: 58 | SEQ ID NO: 12 | SEQ ID NO: 57 | SEQ ID NO: 13 |
| SEQ ID | IL-10RA | IgG2 CH1 domain | IgG2 |
| NO: 23 | SEQ ID NO: 12 | SEQ ID NO: 2 | SEQ ID NO: 14 |
| SEQ ID | IL-10RA | IgG3 CH1 domain | IgG3 |
| NO: 24 | SEQ ID NO: 12 | SEQ ID NO: 3 | SEQ ID NO: 15 |
| SEQ ID | IL-10RA | IgG4 CH1 domain | IgG4 |
| NO: 25 | SEQ ID NO: 12 | SEQ ID NO: 4 | SEQ ID NO: 16 |
| SEQ ID | IL-10RA | IgA1 CH1 domain | IgA1 |
| NO: 26 | SEQ ID NO: 12 | SEQ ID NO: 5 | SEQ ID NO: 17 |
| SEQ ID | IL-10RA | IgA2 CH1 domain | IgA2 |
| NO: 27 | SEQ ID NO: 12 | SEQ ID NO: 6 | SEQ ID NO: 18 |
| SEQ ID | IL-10RA | IgD CH1 domain | IgD |
| NO: 28 | SEQ ID NO: 12 | SEQ ID NO: 7 | SEQ ID NO: 19 |
| SEQ ID | IL-10RA | IgE CH1 domain | IgE |
| NO: 29 | SEQ ID NO: 12 | SEQ ID NO: 8 | SEQ ID NO: 20 |
| SEQ ID | IL-10RA | IgM CH1 domain | IgM |
| NO: 30 | SEQ ID NO: 12 | SEQ ID NO: 9 | SEQ ID NO: 21 |
| SEQ ID | IL-10RA | Albumin | IgG1 |
| NO: 31 | SEQ ID NO: 12 | SEQ ID NO: 10 | SEQ ID NO: 13 |
| SEQ ID | IL-10RA | Casein | IgG1 |
| NO: 32 | SEQ ID NO: 12 | SEQ ID NO: 11 | SEQ ID NO: 13 |
| SEQ ID | mIL-10RA | mIgG1 CH1 domain | mIgG1 |
| NO: 33 | SEQ ID NO: 34 | SEQ ID NO: 35 | SEQ ID NO: 36 |

TABLE 2

| Protein Sequence | Nucleic Acid Sequence |
|---|---|
| SEQ ID NO: 22 | SEQ ID NO: 37 |
| SEQ ID NO: 23 | SEQ ID NO: 38 |
| SEQ ID NO: 24 | SEQ ID NO: 39 |
| SEQ ID NO: 25 | SEQ ID NO: 40 |
| SEQ ID NO: 26 | SEQ ID NO: 41 |
| SEQ ID NO: 27 | SEQ ID NO: 42 |
| SEQ ID NO: 28 | SEQ ID NO: 43 |
| SEQ ID NO: 29 | SEQ ID NO: 44 |
| SEQ ID NO: 30 | SEQ ID NO: 45 |
| SEQ ID NO: 31 | SEQ ID NO: 46 |
| SEQ ID NO: 32 | SEQ ID NO: 47 |

### II. Fusion Protein Production

Methods for producing fusion proteins of the invention are known in the art. For example, DNA molecules encoding a disclosed fusion protein can be chemically synthesized using the sequence information provided herein. Synthetic DNA molecules can be ligated to other appropriate nucleotide sequences, including, e.g., expression control sequences, to produce conventional gene expression constructs encoding the desired fusion protein. Production of defined gene constructs is within routine skill in the art. Exemplary nucleic acid sequences SEQ ID NOs: 37-47, which encode the fusion proteins of SEQ ID NOs: 22-32, can be found in TABLE 2.

Nucleic acids encoding desired fusion proteins can be incorporated (ligated) into expression vectors, which can be introduced into host cells through conventional transfection or transformation techniques. Exemplary host cells are E. coli cells, Chinese hamster ovary (CHO) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the desired fusion protein.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. The gene construct can be introduced into eukaryotic host cells using conventional techniques.

A polypeptide comprising a disclosed fusion protein can be produced by growing (culturing) a host cell transfected with an expression vector encoding such protein, under conditions that permit expression of the polypeptide. Following expression, the polypeptide can be harvested and purified or isolated using techniques known in the art, e.g., affinity tags such as Protein A, Protein G, glutathione-S-transferase (GST) and histidine tags.

### III. Expression Vectors

The fusion proteins of interest may be expressed in a cell of interest by incorporating a gene encoding a fusion protein of interest into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

In certain embodiments, a disclosed expression vector is a viral vector. The terms “viral vector” and “virus” are used interchangeably herein to refer to any of the obligate intracellular parasites having no protein-synthesizing or energy-generating mechanism. The viral genome may be RNA or DNA. The viruses useful in the practice of the present invention include recombinantly modified enveloped or non-enveloped DNA and RNA viruses, preferably selected from baculoviridiae, parvoviridiae, picornoviridiae, herpesviridiae, poxyiridae, or adenoviridiae. The viruses may be modified by recombinant DNA techniques to include expression of exogenous transgenes and may be engineered to be replication deficient, conditionally replicating, or replication competent. Chimeric viral vectors which exploit advantageous elements of each of the parent vector properties (See, e.g., Feng et al. (1997) NATURE BIOTECHNOLOGY 15:866-870) may also be useful in the practice of the present invention. Although it is generally favored to employ a virus from the species to be treated, in some instances it may be advantageous to use vectors derived from different species that possess favorable pathogenic features. For example, equine herpes virus vectors for human gene therapy are described in PCT Publication No. WO 98/27216. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in PCT Publication No. WO 97/06826.

In certain embodiments, the viral vector is an adenovirus. Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses includes the A-F subgenera as well as the individual serotypes thereof, the individual serotypes and A-F subgenera including but not limited to human adenovirus types 1, 2, 3, 4, 4a, 5, 6, 7, 8, 9, 10, 11 (Ad11a and Ad11p), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Preferred are vectors derived from human adenovirus types 2 and 5. Unless stated otherwise, all adenovirus type 5 nucleotide numbers are relative to the NCBI reference sequence AC_000008.1, which is depicted herein in SEQ ID NO: 52.

The adenovirus replication cycle has two phases: an early phase, during which 4 transcription units (E1, E2, E3, and E4) are expressed, and a late phase which occurs after the onset of viral DNA synthesis, and during which late transcripts are expressed primarily from the major late promoter (MLP). The late messages encode most of the virus's structural proteins. The gene products of E1, E2 and E4 are responsible for transcriptional activation, cell transformation, viral DNA replication, as well as other viral functions, and are necessary for viral growth.

The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

### IV. Fusion Protein Modifications

When used as a therapeutic, a fusion protein may be optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into a disclosed fusion protein by DNA shuffling, chain shuffling, CDR shuffling, random mutagenesis and/or site-specific mutagenesis.

Generally, an optimized fusion protein has at least the same, or substantially the same (e.g., at least 85%, 90%, 95%, 96%, 97%, 98%, or 99%) affinity for a ligand as the non-optimized (or parental) fusion protein from which it was derived. Preferably, an optimized fusion protein has a higher affinity for a ligand when compared to a parental fusion protein.

Fusion proteins (e.g., parental and optimized variants) can be engineered to contain certain constant (i.e., Fc) regions with a specified effector function (e.g., antibody-dependent cellular cytotoxicity (ADCC)). Human constant regions are known in the art.

Furthermore, if the fusion protein is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

### V. Methods of Treatment

The foregoing fusion proteins or expression vectors can be used to treat various medical indications. In certain embodiments, the foregoing fusion proteins or expression vectors can be used to treat medical indications that are mediated by a cytokine, for example IL-10. For example, the fusion proteins and expression vectors can be used to treat various cancers or inflammatory diseases.

As used herein, “treat,” “treating” and “treatment” mean the treatment of a disease in a subject, e.g., in a mammal, e.g., in a human This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

In certain embodiments, the fusion proteins and expression vectors disclosed herein can be used to treat various cancers. The cancer cells are exposed to a therapeutically effective amount of the fusion protein or expression vector so as to inhibit or reduce proliferation of the cancer cells. In certain embodiments, administering a therapeutically effective amount of a fusion protein or expression vector to cancer cells reduces IL-10 activity in the cells by at least 30%, at least 40%, at least 50%, at least 60%, at least 70%, at least 80%, at least 90%, or at least 95%. IL-10 activity may be assayed by Western blot as described in Example 2. In some embodiments, a disclosed fusion protein or expression vector can be used to inhibit tumor growth in a subject (e.g., a human patient, also referred to as a human subject), which can be accomplished by administering an effective amount of the fusion protein or expression vector to the subject. In certain embodiments, administering an effective amount of a fusion protein or expression vector to a subject reduces tumor load in that subject by at least 30%, at least 40%, at least 50%, at least 60%, at least 70%, at least 80%, or at least 90%.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments, the cancer is selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, parathyroid cancer (e.g., parathyroid carcinoma), endometrial cancer, neuroendocrine cancer, lymphoma (e.g., Hodgkin and non-Hodgkin), leukemia, merkel cell carcinoma, gastrointestinal stromal tumors, multiple myeloma, uterine cancer, a sarcoma, kidney cancer, ocular cancer, pancreatic cancer, and a germ cell cancer (e.g., ovarian germ cell cancer). In certain embodiments, the cancer may be selected from leukemia, breast cancer, lung cancer, pancreatic cancer, endometrial cancer, ovarian cancer, prostate cancer, cervical cancer, brain cancer, skin cancer, colorectal cancer, gastric cancer, head and neck cancer, and leukemia. In certain embodiments, the cancer is selected from leukemia, breast cancer, cervical cancer, colorectal cancer, lung cancer, pancreatic cancer, prostate cancer, gastric cancer, head and neck cancer, endometrial cancer and ovarian cancer.

In certain embodiments, a fusion protein or expression vector of the disclosure is administered to decrease levels of one or more cytokines in a subject in need thereof (e.g., a subject with an inflammatory condition). In certain embodiments, a disclosed fusion protein or expression vector can be used to treat an inflammatory condition in a subject (e.g., a human subject), which can be accomplished by administering an effective amount of the fusion protein or expression vector to the subject.

As used herein, an inflammatory condition is a disease or condition characterized, in whole or in part, by inflammation or an inflammatory response in the patient. Inflammatory conditions treatable using the fusion proteins or expression vectors of the invention may be characterized, for example, based on the primary tissue affected, the mechanism of action underlying the condition, or the portion of the immune system that is misregulated or overactive. In certain embodiments, examples of inflammatory conditions that may be treated include inflammation of the lungs (e.g., asthma, adult respiratory distress syndrome, bronchitis, pulmonary inflammation, pulmonary fibrosis, and cystic fibrosis), joints (e.g., rheumatoid arthritis, rheumatoid spondylitis, juvenile rheumatoid arthritis, osteoarthritis, gouty arthritis and other arthritic conditions), connective tissue, eyes (e.g., uveitis (including iritis), conjunctivitis, scleritis, and keratoconjunctivitis sicca), nose, bowel (e.g., Crohn's disease, ulcerative colitis, inflammatory bowel disease, inflammatory bowel syndrome, and distal proctitis), kidney (e.g., glomerulonephritis, interstitial nephritis, lupus nephritis, nephritis secondary to Wegener's disease, acute renal failure secondary to acute nephritis, Goodpasture's syndrome, post-obstructive syndrome and tubular ischemia), liver (e.g., hepatitis (arising from viral infection, autoimmune responses, drug treatments, toxins, environmental agents, or as a secondary consequence of a primary disorder), obesity, biliary atresia, primary biliary cirrhosis and primary sclerosing cholangitis), skin (e.g., psoriasis, eczema, and dermatitis, e. g., eczematous dermatitides, topic and seborrheic dermatitis, allergic or irritant contact dermatitis, eczema craquelee, photoallergic dermatitis, phototoxicdermatitis, phytophotodermatitis, radiation dermatitis, and stasis dermatitis), central nervous system (e.g., multiple sclerosis and neurodegenerative diseases such as Alzheimer's disease, Parkinson's disease or dementia associated with HIV infection), vascular system (e.g. coronary infarct damage, peripheral vascular disease, myocarditis, vasculitis, revascularization of stenosis, atherosclerosis, and vascular disease associated with Type II diabetes), endocrine system (e.g., autoimmune thyroiditis (Hashimoto's disease), Type I diabetes, inflammation in liver and adipose tissue associated with Type II diabetes, and acute and chronic inflammation of the adrenal cortex) heart, or adipose tissue. The disclosure contemplates that some inflammatory conditions involve inflammation in multiple tissues. Moreover, the disclosure contemplates that some inflammatory conditions may fall into multiple categories. In certain embodiments, the inflammatory condition is an autoimmune disease. Exemplary autoimmune diseases include, but are not limited to, rheumatoid arthritis, psoriasis (including plaque psoriasis), psoriatic arthritis, ankylosing spondylitis, ulcerative colitis, multiple sclerosis, lupus, alopecia, autoimmune pancreatitis, Celiac disease, Behcet's disease, Cushing syndrome, and Grave's disease. In certain embodiments, the inflammatory condition is a rheumatoid disorder. Exemplary rheumatoid disorders include, but are not limited to, rheumatoid arthritis, juvenile arthritis, bursitis, spondylitis, gout, scleroderma, Still's disease, and vasculitis. It is noted that certain categories of conditions overlap. For example, rheumatoid arthritis is an inflammatory rheumatoid disorder, an inflammatory joint disorder, and an autoimmune disorder.

The term “effective amount” as used herein refers to the amount of an active component (e.g., the amount of a fusion protein or expression vector of the present invention) sufficient to effect beneficial or desired results. An effective amount can be administered in one or more administrations, applications or dosages and is not intended to be limited to a particular formulation or administration route.

In certain embodiments, a therapeutically effective amount of a fusion protein is in the range of 0.1 mg/kg to 100 mg/kg, e.g., 1 mg/kg to 100 mg/kg, 1 mg/kg to 10 mg/kg, 1 mg/kg to 5 mg/kg, 10 mg/kg, 7.5 mg/kg, 5 mg/kg, or 2.5 mg/kg. In certain embodiments, a therapeutically effective amount of an expression vector, e.g., a recombinant virus, is in the range of 10² to 10¹⁵ plaque forming units (pfus), e.g., 10² to 10¹⁰. 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the patient, the in vivo potency of the fusion protein or expression vector, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. Formulation of fusion protein- or expression vector-based drugs is within ordinary skill in the art. In some embodiments, a fusion protein or expression vector is lyophilized, and then reconstituted in buffered saline, at the time of administration.

For therapeutic use, a fusion protein or expression vector preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing fusion proteins or expression vectors disclosed herein can be presented in a dosage unit form and can be prepared by any suitable method. A pharmaceutical composition should be formulated to be compatible with its intended route of administration. Examples of routes of administration are intravenous (IV), intradermal, inhalation, intraocular, intranasal, transdermal, topical, transmucosal, and rectal administration.

A preferred route of administration for fusion proteins is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, a delivery vehicle (e.g., a recombinant virus) and/or a therapeutic agent of the invention is administered in combination with a checkpoint inhibitor, e.g., an anti-CTLA-4 antibody, an anti-PD-1 antibody, or an anti-PD-L1 antibody. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies include, for example, atezolizumab (Tecentriq®, Genentech), duvalumab (AstraZeneca), MEDI4736, avelumab (Bavencio , EMD Serono), and BMS 936559 (Bristol Myers Squibb Co.).

The term administered “in combination,” as used herein, is understood to mean that two (or more) different treatments are delivered to the subject during the course of the subject's affliction with the disorder, such that the effects of the treatments on the subject overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Throughout the description, where compositions, devices, and systems are described as having, including, or comprising specific components, or where processes and methods are described as having, including, or comprising specific steps, it is contemplated that, additionally, there are compositions, devices, and systems of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular virus, that virus can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1: IL-10RA Fusion Protein Plasmid Construction

This Example describes the production of plasmids and viral expression vectors that encode IL-10RA fusion proteins.

Nucleotide sequences encoding a series of human IL-10RA fusion proteins were generated. A first fusion protein, hIL-10R-IgG1 (SEQ ID NO: 58), included residues 1-229 of human IL-10RA (ending in SLTRQ), immediately followed by residues 84-330 of the human IgG1 sequence, (beginning with NVNHK). A second fusion protein, hIL-10R-Fc (SEQ ID NO: 48), included residues 1-235 of human IL-10RA (ending in FTVTN), immediately followed by residues 104-324 of human IgG1 (beginning at DKTHT). Details of the fusion proteins are shown in TABLE 3.

TABLE 3

| Fusion | hIL-10RA | hIgG1 | hIL-10RA-hIgG1 |
|---|---|---|---|
| Protein | Residues | Residues | Junction |
| hIL-10R-IgG1 | 1-229 | 84-330 | SLTRQ-NVNHKPSNTKVDKRVEPKSCDKT |
| hIL-10R-Fc | 1-235 | 104-324 | SLTRQYFTVTN-DKTHT |

Nucleotide sequences encoding the fusion proteins were cloned into plasmids for downstream applications as appropriate. In particular, recombinant adenoviral vectors were generated that expressed no transgene, hIL-10R-IgG1, or hIL-10RA-Fc.

### Example 2: IL-10R Fusion Protein Activity

A549 cells (human lung cancer cells) were infected with viral vectors expressing no transgene, hIL-10R-IgG1, or hIL-10RA-Fc, as described in Example 1, at 10 MOI, and cultured for four days. Conditioned media from the cell culture was collected and THP-1 cells (human leukemic monocytes) were suspended in the conditioned media at 5×10⁶ cells/ml. Cells were either treated with human IL-10 at 50 ng/ml at 37° C. for 30 minutes or kept as controls. To assay for IL-10 activity, extracted cellular protein from the THP-1 cells was probed by Western blot for phosphorylated Stat3. Total Stat3 was used as a loading control.

IL-10 induced Stat3 phosphorylation in THP-1 cells cultured in conditioned media from cells infected with viral vectors expressing no transgene or hIL-10RA-Fc. However, IL-10 did not induce Stat3 phosphorylation in THP-1 cells cultured in conditioned media from cells infected with hIL10R-IgG-expressing virus. These results demonstrate that the hIL-10R-IgG1 fusion protein blocked IL-10 from activating the Stat3 signaling cascade.

### INCORPORATION BY REFERENCE

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

### Additional Aspects

Aspect 1. An isolated fusion protein comprising:
(i) a portion of an IL-10 receptor; and
(ii) an amino acid linker; and at least one of
(iii) a portion of an extracellular domain, transmembrane domain, or intracellular domain of a cytokine, cytokine receptor, or immunomodulatory protein;
(iv) an immunoglobulin (Ig) hinge region; or
(v) an immunoglobulin (Ig) Fc domain.

Aspect 2. The isolated fusion protein of Aspect 1, wherein the linker comprises from about 5 to about 40 amino acid residues.

Aspect 3. An isolated fusion protein comprising, in an N- to C-terminal orientation:
(i) a soluble portion of an extracellular domain of an IL-10 receptor;
(ii) an amino acid linker;
(iii) an immunoglobulin (Ig) hinge region; and
(iv) an immunoglobulin (Ig) Fc domain;
   wherein the linker comprises from about 5 to about 40 amino acid residues.

Aspect 4. The isolated fusion protein of any one of Aspects 1-3, wherein the linker comprises from about 5 to about 30 amino acid residues.

Aspect 5. The isolated fusion protein of any one of Aspects 1-3, wherein the linker comprises from about 5 to about 20 amino acid residues.

Aspect 6. The isolated fusion protein of any one of Aspects 1-3, wherein the linker comprises from about 5 to about 15 amino acid residues.

Aspect 7. The isolated fusion protein of any one of Aspects 1-3, wherein the linker comprises from about 10 to about 40 amino acid residues.

Aspect 8. The isolated fusion protein of any one of Aspects 1-3, wherein the linker comprises from about 10 to about 30 amino acid residues.

Aspect 9. The isolated fusion protein of any one of Aspects 1-3, wherein the linker comprises from about 10 to about 20 amino acid residues.

Aspect 10. The isolated fusion protein of any one of Aspects 1-3, wherein the linker comprises from about 10 to about 15 amino acid residues.

Aspect 11. The isolated fusion protein of any one of Aspects 1-10, wherein the linker comprises a sequence derived from an endogenous human protein.

Aspect 12. The isolated fusion protein of any one of Aspects 1-11, wherein the linker comprises a C-terminal portion of an immunoglobulin (Ig) CH1 domain.

Aspect 13. The isolated fusion protein of Aspect 12, wherein the Ig CH1 domain is selected from an IgG1, IgG2, IgG3, IgG4, IgA1, IgA2, IgD, IgE, and IgM CH1 domain.

Aspect 14. The isolated fusion protein of Aspect 13, wherein the linker comprises an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 53, SEQ ID NO: 54, and SEQ ID NO: 57.

Aspect 15. The isolated fusion protein of Aspect 14, wherein the Ig CH1 domain is an IgG1 CH1 domain.

Aspect 16. The isolated fusion protein of Aspect 15, wherein the linker comprises an amino acid sequence selected from SEQ ID NO: 1, SEQ ID NO: 53, SEQ ID NO: 54, and SEQ ID NO: 57.

Aspect 17. The isolated fusion protein of Aspect 16, wherein the linker comprises the amino acid sequence of SEQ ID NO: 57.

Aspect 18. The isolated fusion protein of any one of Aspects 1-11, wherein the linker comprises a sequence derived from a human protein selected from albumin and casein.

Aspect 19. The isolated fusion protein of Aspect 18, wherein the linker comprises an amino acid sequence selected from SEQ ID NO: 10 and SEQ ID NO: 11.

Aspect 20. The isolated fusion protein of any one of Aspects 1-11, wherein the linker comprises a sequence derived from a cytokine, signaling molecule, immunomodulatory protein or a peptide.

Aspect 21. The isolated fusion protein of any one of Aspects 1-20, wherein the linker comprises a cleavage site.

Aspect 22. The isolated fusion protein of Aspect 21, wherein the cleavage site is a proteolytic cleavage site.

Aspect 23. The isolated fusion protein of Aspect 22, wherein the proteolytic cleavage site is cleaved by a protease that is present in the endoplasmic reticulum or golgi of a eukaryotic cell.

Aspect 24. The isolated fusion protein of Aspects 22 or 23, wherein the proteolytic cleavage site is a furin cleavage site.

Aspect 25. The isolated fusion protein of Aspect 24, wherein the furin cleavage site comprises RX₁X₂R (SEQ ID NO: 50), wherein X₁ is any amino acid, and X₂ is Lys or Arg.

Aspect 26. The isolated fusion protein of Aspect 22, wherein the furin cleavage site comprises RAKR (SEQ ID NO: 51).

Aspect 27. The isolated fusion protein of any one of Aspects 1-20, wherein the linker comprises an amino acid sequence which is proteolytically stable in a mammal or plant.

Aspect 28. The isolated fusion protein of any one of Aspects 1-27, wherein the IL-10 receptor is a human IL-10 receptor.

Aspect 29. The isolated fusion protein of Aspect 28, wherein the soluble portion of an extracellular domain of an IL-10 receptor comprises the amino acid sequence of SEQ ID NO: 12.

Aspect 30. The isolated fusion protein of Aspect 28, wherein the soluble portion of an extracellular domain of an IL-10 receptor comprises amino acid residues 22-229 of SEQ ID NO: 12.

Aspect 31. The isolated fusion protein of any one of Aspects 1-30, wherein the Ig Fc domain and hinge region are selected from a human IgG1, IgG2, IgG3, IgG4, IgA1, IgA2, IgD, IgE, and IgM Fc domain and hinge region.

Aspect 32. The isolated fusion protein of Aspect 31, wherein the Ig Fc domain and hinge region comprise an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

Aspect 33. The isolated fusion protein of Aspect 32, wherein the Ig Fc domain and hinge region are a human IgG1 Fc domain and hinge region.

Aspect 34. The isolated fusion protein of Aspect 33, wherein the Ig Fc domain and hinge region comprise the amino acid sequence of SEQ ID NO: 13.

Aspect 35. The isolated fusion protein of any one of Aspects 1-34, wherein the Ig Fc, Ig hinge region, and Ig CH1 domain are derived from a single immunoglobulin.

Aspect 36. The isolated fusion protein of any one of Aspects 1-35, wherein the fusion protein comprises an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 55, SEQ ID NO: 56, and SEQ ID NO: 58.

Aspect 37. The isolated fusion protein of Aspect 36, wherein the fusion protein comprises an amino acid sequence selected from SEQ ID NO: 22, SEQ ID NO: 55, SEQ ID NO: 56, and SEQ ID NO: 58.

Aspect 38. The isolated fusion protein of Aspect 37, wherein the fusion protein comprises the amino acid sequence of SEQ ID NO: 58.

Aspect 39. A cytokine binding protein comprising two fusion proteins of any one of Aspects 1-38, wherein each fusion protein comprises an extracellular domain of a cytokine receptor, wherein the two fusion proteins are covalently linked together, and wherein the two extracellular domains together define a binding site for binding a cytokine.

Aspect 40. An isolated nucleic acid comprising a nucleotide sequence encoding the fusion protein of any one of Aspects 1-39.

Aspect 41. An expression vector comprising the nucleic acid of Aspect 40.

Aspect 42. A host cell comprising the expression vector of any one of Aspect 41.

Aspect 43. A method of producing a fusion protein comprising:
(a) growing the host cell of Aspect 42 under conditions to express the fusion protein; and
(b) purifying the fusion protein.

Aspect 44. A pharmaceutical composition comprising: (i) the fusion protein of any one of Aspects 1-39 or the expression vector of Aspect 41; and (ii) at least one pharmaceutically acceptable carrier or diluent.

Aspect 45. A method of expressing a fusion protein in a target cell comprising exposing the cell to an effective amount of the expression vector of Aspect 41 to express the fusion protein.

Aspect 46. The method of Aspect 45, wherein the fusion protein is cleaved posttranslationally into two polypeptide chains.

Aspect 47. A method of inhibiting proliferation of a tumor cell comprising exposing the cell to an effective amount of the dimeric fusion protein of Aspect 39 to inhibit proliferation of the tumor cell.

Aspect 48. A method of inhibiting proliferation of a tumor cell comprising exposing the cell to an effective amount of the fusion protein of any one of Aspects 1-38 to inhibit proliferation of the tumor cell.

Aspect 49. A method of inhibiting tumor growth in a subject in need thereof, the method comprising administering to the subject to an effective amount of the dimeric fusion protein of Aspect 39 to inhibit growth of the tumor.

Aspect 50. A method of inhibiting tumor growth in a subject in need thereof, the method comprising administering to the subject to an effective amount of the fusion protein of any one of Aspects 1-38 to inhibit growth of the tumor.

Aspect 51. A method of treating cancer in a subject in need thereof, the method comprising administering an effective amount of the dimeric fusion protein of Aspect 39 to the subject.

Aspect 52. A method of treating cancer in a subject in need thereof, the method comprising administering an effective amount of the fusion protein of any one of Aspects 1-38 to the subject.

Aspect 53. A method of reducing IL-10 activity in a cell comprising exposing the cell to an effective amount of the dimeric fusion protein of Aspect 39 to reduce IL-10 activity.

Aspect 54. A method of reducing IL-10 activity in a cell comprising exposing the cell to an effective amount of the fusion protein of any one of Aspects 1-38 to reduce IL-10 activity.

Aspect 55. A method of treating an inflammatory condition in a subject in need thereof, the method comprising administering an effective amount of the dimeric fusion protein of Aspect 39 to the subject.

Aspect 56. A method of treating an inflammatory condition in a subject in need thereof, the method comprising administering an effective amount of the fusion protein of any one of Aspects 1-38 to the subject.

Aspect 57. A method of inhibiting proliferation of a tumor cell comprising exposing the cell to an effective amount of the expression vector of Aspect 41 to inhibit proliferation of the tumor cell.

Aspect 58. A method of inhibiting tumor growth in a subject in need thereof, the method comprising administering to the subject an effective amount of the expression vector of Aspect 41 to inhibit growth of the tumor.

Aspect 59. A method of treating cancer in a subject in need thereof, the method comprising administering an effective amount of the expression vector of Aspect 41 to the subject.

Aspect 60. A method of reducing IL-10 activity in a cell comprising exposing the cell to an effective amount of the expression vector of Aspect 41 to reduce IL-10 activity.

Aspect 61. A method of treating an inflammatory condition in a subject in need thereof, the method comprising administering an effective amount of the expression vector of Aspect 41 to the subject.

Aspect 62. A method of treating an infection in a subject in need thereof, the method comprising administering an effective amount of the expression vector of Aspect 41 to the subject.

Aspect 63. The method of any one of Aspects 51, 52, or 59, wherein the cancer is selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, parathyroid cancer (e.g., parathyroid carcinoma), endometrial cancer, neuroendocrine cancer, lymphoma (e.g., Hodgkin and non-Hodgkin), leukemia, merkel cell carcinoma, gastrointestinal stromal tumors, multiple myeloma, uterine cancer, a sarcoma, kidney cancer, ocular cancer, pancreatic cancer, and a germ cell cancer (e.g., ovarian germ cell cancer).

Aspect 64. The method of any one of Aspects 51, 52, or 59, wherein the cancer is selected from leukemia, breast cancer, lung cancer, pancreatic cancer, endometrial cancer, ovarian cancer, prostate cancer, cervical cancer, brain cancer, skin cancer, colorectal cancer, gastric cancer, head and neck cancer, and leukemia.

Aspect 65. The method of Aspect 64, wherein the cancer is selected from skin cancer, head and neck cancer, and lung cancer.

Aspect 66. The method of any one of Aspects 49-52, 55, 56, 58, 59, or 61-65, wherein the fusion protein or expression vector is administered to the subject in combination with one or more therapies selected from surgery, radiation, chemotherapy, immunotherapy, hormone therapy, and virotherapy.

Aspect 67. The method of any one of Aspects 49-52, 55, 56, 58, 59, or 61-65, wherein the fusion protein or expression vector is administered to the subject in combination with a lymphocyte.

Aspect 68. The method of Aspect 67, wherein the lymphocyte is a T-cell.

Aspect 69. The method of Aspect 68, wherein the T-cell is a CAR T-cell.

Aspect 70. The method of any one of Aspects 49-52, 55, 56, 58, 59, or 61-69, wherein the subject is a human or animal.

Aspect 71. The method of Aspect 70, wherein the subject is a pediatric human.

What is claimed is:

1. A method of expressing a fusion protein in a target cell comprising exposing the cell to an effective amount of an expression vector to express the fusion protein, wherein the expression vector comprises a nucleic acid comprising a nucleotide sequence encoding a fusion protein comprising, in an N- to C-terminal orientation:
(i) a soluble portion of an extracellular domain of a human IL-10 receptor subunit alpha;
(ii) an amino acid linker;
(iii) an immunoglobulin (Ig) hinge region; and
(iv) an immunoglobulin (Ig) Fc domain;
wherein the linker consists of 10 to 40 amino acid residues, wherein the linker is a sequence derived from an endogenous human protein.

2. The method of claim 1, wherein the linker comprises a sequence derived from a human protein selected from albumin and casein, or comprises a C-terminal portion of an immunoglobulin (Ig) CH1 domain having greater than 90% sequence identity to an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 53, SEQ ID NO: 57, SEQ ID NO: 59, SEQ ID NO: 60, SEQ ID NO: 61, SEQ ID NO: 62, and SEQ ID NO: 63.

3. The method of claim 1, wherein the linker comprises an amino acid sequence which is proteolytically stable in a mammal or plant or comprises a cleavage site.

4. The method of claim 1, wherein the soluble portion of an extracellular domain of a human IL-10 receptor has greater than 95% sequence identity to amino acid residues 22-229 of SEQ ID NO: 12.

5. The method of claim 4, wherein the soluble portion of an extracellular domain of an IL-10 receptor comprises the amino acid sequence of SEQ ID NO: 12 or amino acid residues 22-229 of SEQ ID NO: 12.

6. The method of claim 1, wherein the Ig hinge region and the Ig Fc domain together comprise an amino acid sequence having greater than 95% identity to SEQ ID NOs: 13-21

7. The method of claim 6, wherein the Ig Fc domain and Ig hinge region together comprise an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

8. The method of claim 1, wherein the fusion protein has greater than 95% sequence identity to an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 55, and SEQ ID NO: 58.

9. The method of claim 1, wherein the fusion protein is cleaved posttranslationally into two polypeptide chains.

10. A method comprising exposing a cell to an effective amount of a dimeric fusion protein, wherein (i) the cell is a tumor cell, the method is for inhibiting proliferation, and the effective amount inhibits proliferation of the tumor cell, or (ii) the method is for reducing IL-10 activity in the cell, and the effective amount reduces IL-10 activity in the cell, wherein the fusion protein comprises, in an N- to C-terminal orientation:
(i) a soluble portion of an extracellular domain of a human IL-10 receptor subunit alpha;
(ii) an amino acid linker;
(iii) an immunoglobulin (Ig) hinge region; and
(iv) an immunoglobulin (Ig) Fc domain;
wherein the linker consists of 10 to 40 amino acid residues, wherein the linker is a sequence derived from an endogenous human protein.

11. The method of claim 10, wherein the dimeric fusion protein is administered by administering an expression vector to express the fusion protein, wherein the expression vector comprises a nucleic acid comprising a nucleotide sequence encoding the fusion protein.

12. A method comprising administering to a subject an effective amount of a fusion protein, wherein (i) the method is for inhibiting tumor growth in the subject who is in need thereof, and the effective amount inhibits tumor growth in the subject, (ii) the method is for treating cancer in the subject who is in need thereof, or (iii) the method is for treating an inflammatory condition in the subject who is in need thereof, wherein the fusion protein comprises, in an N- to C-terminal orientation:
(i) a soluble portion of an extracellular domain of a human IL-10 receptor subunit alpha;
(ii) an amino acid linker;
(iii) an immunoglobulin (Ig) hinge region; and
(iv) an immunoglobulin (Ig) Fc domain;
wherein the linker consists of 10 to 40 amino acid residues, wherein the linker is a sequence derived from an endogenous human protein.

13. The method of claim 12, wherein the cancer is selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, parathyroid cancer, endometrial cancer, neuroendocrine cancer, lymphoma, leukemia, merkel cell carcinoma, gastrointestinal stromal tumors, multiple myeloma, uterine cancer, a sarcoma, kidney cancer, ocular cancer, pancreatic cancer, and a germ cell cancer.

14. The method of claim 12, wherein the cancer is selected from skin cancer, head and neck cancer, and lung cancer

15. The method of claim 12, wherein the fusion protein or expression vector is administered to the subject in combination with one or more therapies selected from surgery, radiation, chemotherapy, immunotherapy, hormone therapy, and virotherapy.

16. The method of claim 12, wherein the fusion protein or expression vector is administered to the subject in combination with a lymphocyte, optionally a T-cell, optionally a CAR T-cell.

17. The method of claim 12, wherein the subject is a human, optionally a pediatric human, or animal.

18. The method of claim 12, wherein the fusion protein is administered by administering an expression vector to express the fusion protein, wherein the expression vector comprises a nucleic acid comprising a nucleotide sequence encoding the fusion protein.

19. A cytokine binding protein comprising two fusion proteins comprising, in an N- to C-terminal orientation:
(i) a soluble portion of an extracellular domain of a human IL-10 receptor subunit alpha;
(ii) an amino acid linker;
(iii) an immunoalobulin (Ig) hinge region; and
(iv) an immunoglobulin (Ig) Fc domain;
wherein the linker consists of 10 to 40 amino acid residues, wherein the linker is a sequence derived from an endogenous human protein, wherein the two fusion proteins are covalently linked together, and wherein the two extracellular domains together define a binding site for binding a eytokine.


SEQUENCE LISTING (64 sequences)

SEQ ID NO: 1 - protein, 12 aa, Homo sapiens
KPSNTKVDKKVE

SEQ ID NO: 2 - protein, 12 aa, Homo sapiens
KPSNTKVDKTVE

SEQ ID NO: 3 - protein, 12 aa, Homo sapiens
KPSNTKVDKRVE

SEQ ID NO: 4 - protein, 12 aa, Homo sapiens
KPSNTKVDKRVE

SEQ ID NO: 5 - protein, 12 aa, Homo sapiens
VKHYTNPSQDVT

SEQ ID NO: 6 - protein, 12 aa, Homo sapiens
VKHYTNPSQDVT

SEQ ID NO: 7 - protein, 10 aa, Homo sapiens
TASKSKKEIF

SEQ ID NO: 8 - protein, 14 aa, Homo sapiens
TPSSTDWVDNKTFS

SEQ ID NO: 9 - protein, 13 aa, Homo sapiens
VQHPNGNKEKNVP

SEQ ID NO: 10 - protein, 13 aa, Homo sapiens
VQHPNGNKEKNVP

SEQ ID NO: 11 - protein, 12 aa, Homo sapiens
REKQTDEIKDTR

SEQ ID NO: 12 - protein, 229 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQ

SEQ ID NO: 13 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 227 aa, Homo sapiens
RKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 15 - protein, 278 aa, Homo sapiens
LKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 16 - protein, 228 aa, Homo sapiens
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 255 aa, Homo sapiens
VPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 18 - protein, 242 aa, Homo sapiens
VPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 19 - protein, 286 aa, Homo sapiens
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 20 - protein, 325 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 21 - protein, 351 aa, Homo sapiens
LPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 22 - protein, 472 aa, synthetic construct
  REGION 1..472: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 468 aa, synthetic construct
  REGION 1..468: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 24 - protein, 519 aa, synthetic construct
  REGION 1..519: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 25 - protein, 469 aa, synthetic construct
  REGION 1..469: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 496 aa, synthetic construct
  REGION 1..496: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 27 - protein, 483 aa, synthetic construct
  REGION 1..483: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 28 - protein, 525 aa, synthetic construct
  REGION 1..525: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 29 - protein, 568 aa, synthetic construct
  REGION 1..568: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 30 - protein, 593 aa, synthetic construct
  REGION 1..593: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 31 - protein, 472 aa, synthetic construct
  REGION 1..472: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYVPKEFNAETFTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 472 aa, synthetic construct
  REGION 1..472: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQREKQTDEIKDTRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 467 aa, synthetic construct
  REGION 1..467: note = Synthetic
MLSRLLPFLVTISSLSLEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 232 aa, Mus musculus
MLSRLLPFLVTISSLSLEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQ

SEQ ID NO: 35 - protein, 12 aa, Mus musculus
STKVDKKIVPRD

SEQ ID NO: 36 - protein, 223 aa, Mus musculus
CGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 37 - DNA, 1419 bp, synthetic construct
  misc_feature 1..1419: note = Synthetic
  source 1..1419: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - DNA, 1404 bp, synthetic construct
  misc_feature 1..1404: note = Synthetic
  source 1..1404: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 39 - DNA, 1557 bp, synthetic construct
  misc_feature 1..1557: note = Synthetic
  source 1..1557: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagaagcccagcaacaccaaggtggacaagagagttgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 40 - DNA, 1407 bp, synthetic construct
  misc_feature 1..1407: note = Synthetic
  source 1..1407: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 41 - DNA, 1488 bp, synthetic construct
  misc_feature 1..1488: note = Synthetic
  source 1..1488: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcaggtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgagcgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 42 - DNA, 1449 bp, synthetic construct
  misc_feature 1..1449: note = Synthetic
  source 1..1449: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcaggtgaagcactacacgaatcccagccaggatgtgactgtgccctgcccagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcatggcgggtaaacccacccatgtcaatgtgtctgttgtcatggcggaggtggacggcacctgctac

SEQ ID NO: 43 - DNA, 1575 bp, synthetic construct
  misc_feature 1..1575: note = Synthetic
  source 1..1575: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagaccgccagcaagagtaagaaggagatcttccgctggccagagtctccaaaggcacaggcctcctcagtgcccactgcacaaccccaagcagagggcagcctcgccaaggcaaccacagccccagccaccacccgtaacacaggaagaggaggagaagagaagaagaaggagaaggagaaagaggaacaagaagagagagagacaaagacaccagagtgtccgagccacacccagcctcttggcgtctacctgctaacccctgcagtgcaggacctgtggctccgggacaaagccaccttcacctgcttcgtggtgggcagtgacctgaaggatgctcacctgacctgggaggtggccgggaaggtccccacagggggcgtggaggaagggctgctggagcggcacagcaacggctcccagagccagcacagccgtctgaccctgcccaggtccttgtggaacgcggggacctccgtcacctgcacactgaaccatcccagcctcccaccccagaggttgatggcgctgagagaacccgctgcgcaggcacccgtcaagctttccctgaacctgctggcctcgtctgaccctcccgaggcggcctcgtggctcctgtgtgaggtgtctggcttctcgccccccaacatcctcctgatgtggctggaggaccagcgtgaggtgaacacttctgggtttgcccccgcacgcccccctccacagcccgggagcaccacgttctgggcctggagtgtgctgcgtgtcccagccccgcccagccctcagccagccacctacacgtgtgtggtcagccacgaggactcccggactctgctcaacgccagccggagcctagaagtcagctatgtaacagaccatggccccatgaaa

SEQ ID NO: 44 - DNA, 1704 bp, synthetic construct
  misc_feature 1..1704: note = Synthetic
  source 1..1704: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagactccatcgtccacagactgggtcgacaacaaaaccttcagcgtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaa

SEQ ID NO: 45 - DNA, 1779 bp, synthetic construct
  misc_feature 1..1779: note = Synthetic
  source 1..1779: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcaggtccagcaccccaacggcaacaaagaaaagaacgtgcctcttccagtgattgctgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccggtaaacccaccctgtacaacgtgtccctggtcatgtccgacacagctggcacctgctac

SEQ ID NO: 46 - DNA, 1416 bp, synthetic construct
  misc_feature 1..1416: note = Synthetic
  source 1..1416: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtacgttcccaaagagtttaatgctgaaacattcacccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - DNA, 1416 bp, synthetic construct
  misc_feature 1..1416: note = Synthetic
  source 1..1416: mol_type = other DNA
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagagagaaaaacagactgatgaaatcaaggatactaggcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 462 aa, synthetic construct
  REGION 1..462: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 15 aa, synthetic construct
  REGION 1..15: note = Synthetic
GGGGSGGGGSGGGGS

SEQ ID NO: 50 - skipped

SEQ ID NO: 51 - protein, 4 aa, synthetic construct
  REGION 1..4: note = Furin cleavage site
RAKR

SEQ ID NO: 52 - DNA, 35938 bp, Adenovirus type 5
  source 1..35938: mol_type = unassigned DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 53 - protein, 10 aa, Homo sapiens
SNTKVDKKVE

SEQ ID NO: 54 - protein, 8 aa, Homo sapiens
TKVDKRVE

SEQ ID NO: 55 - protein, 470 aa, synthetic construct
  REGION 1..470: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 468 aa, synthetic construct
  REGION 1..468: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 16 aa, Homo sapiens
NVNHKPSNTKVDKRVE

SEQ ID NO: 58 - protein, 476 aa, synthetic construct
  REGION 1..476: note = Synthetic
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 14 aa, Homo sapiens
NHKPSNTKVDKRVE

SEQ ID NO: 60 - protein, 12 aa, Homo sapiens
KPSNTKVDKRVE

SEQ ID NO: 61 - protein, 10 aa, Homo sapiens
SNTKVDKRVE

SEQ ID NO: 62 - protein, 16 aa, Homo sapiens
NVNHKPSNTKVDKKVE

SEQ ID NO: 63 - protein, 14 aa, Homo sapiens
NHKPSNTKVDKKVE

SEQ ID NO: 64 - protein, 8 aa, Homo sapiens
TKVDKKVE